(19) United States
(12) Patent Application Publication
(10) Pub. No.: US 2007/0173463 A1
(43) Pub. Date: Jul. 26, 2007

(54) Method and composition for treating otitis media

(76) Inventor: Paul B. Brechtelsbauer, Greenville, NC (US)

Correspondence Address: BURNS & LEVINSON LLP, 1700 K STREET, NW, SUITE 720, WASHINGTON, DC, 20006, US

(21) Appl. No.: 11/336,325

(22) Filed: Jan. 20, 2006

(51) Int. Cl. A61K 31/7034; A61K 31/573; A61K 31/496; A61K 31/137; A61K 31/7048; A61K 31/545

(52) U.S. Cl. 514/35; 514/37; 514/39; 514/649; 514/200; 514/253.08; 514/171; 514/28

(57) ABSTRACT

A combination of agents for use at the time of placement of tubes in the eardrums is presented specifically to address both the issue of infection and the issue of tube obstruction from blood. Tube placement for chronic fluid behind the eardrum or recurrent ear infections is one of the most commonly performed procedures. It is not uncommon for a small amount of blood to accumulate and block a tube placed in the eardrum. The eardrop formulation disclosed herein contains any of various antibiotics which have heretofore been placed at the time of tube placement plus a vasoconctrictor not only to treat infection and reduce the likelihood of chronic drainage from the tube and therefore ear drainage but also to prevent or minimize bleeding at or near the incision made to place a tube.

### FIELD OF INVENTION

The present invention relates to treatment of ear infections. It relates more specifically to the treatment of otitis media and the accumulation of fluid in the middle ear that is often consequent to chronic or repeated ear infections in the young. Most particularly, it relates to the use of myringotomy with tube, the placement of a drainage tube through a small puncture in the eardrum to drain the accumulation of fluid in the middle ear.

### BACKGROUND OF THE INVENTION

Recurrent ear infections are a chronic problem in many preschool children and sometimes even in older children and adults. Recurrent infections typically occur in the middle ear, and buildup of middle ear fluid is a common complication of such infections. Middle ear infections and consequent fluid buildup occur most often as a result of inadequate eustachian tube function. This accumulation of fluid in the middle ear space that results from a poorly functioning eustachian tube both results from and facilitates further infection of that space. Even when a middle ear infection has been treated by antibiotics, fluid buildup can remain for weeks or months and can have serious adverse affects on hearing function.

Ear infections are commonly treated with oral antibiotics. However, in many cases the infections are numerous or the fluid does not clear with treatment. When fluid buildup is chronic or long lasting, usually the relevant eustachian tube is not functioning properly. In this case, the clinician will likely recommend placement of a tube in the affected eardrum. A tube in the eardrum allows the middle ear to drain and ventilate by an alternate pathway, the ear canal. Providing this ventilation has been shown to reduce the rate of infections and allows for resolution of chronic fluid.

In order to place a tube in the eardrum, the surgeon typically views the deep portion of the ear canal through a microscope. An opening is made in the eardrum with a small knife or a pick. The opening allows any fluid trapped in the middle ear behind the eardrum to be removed with a small suction. Any of a number of available tubes is then placed in the opening. Typically the tube is a biocompatible plastic grommet.

Commonly the eardrum bleeds at the site of the incision. It is rare for the quantity of blood to be large, but any continued bleeding after placement of the tube creates risk of obstruction of the tube by a blood clot. At the time of tube placement, most surgeons apply topical antibiotic drops not only to treat infection but to attempt to maintain patency of the tube which has been placed. Unfortunately, the antibiotic does not necessarily counteract this tendency for bleeding in the post-operative period, and the tube may become occluded with blood. Often, further topical drops of various types may be used in order to attempt to open a blocked tube. These conservative measures often fail. Since the tube is essentially ineffective when blocked, this result often means further ear infections, more visits to the doctor, and possibly a return visit to the operating room for a new tube.

Drainage, that is, fluid oozing or seeping from the middle ear, most often as a result of infection, is a fairly common problem after tube placement, occurring in approximately one third of cases. However, placement of a tube in the eardrum also provides a new route for applying medications that reach the middle ear space. Specifically, medications may be applied to the eardrum directly as a topical preparation. It is has been shown that placement of antibiotic eardrops (with or without a steroid in combination) reduces the probability of drainage of the ear following tube placement.

Unfortunately, as a result of inflammation in the middle ear, the blood vessels of the middle ear and eardrum often become dilated and have a tendency to bleed when and after an incision is made at the time of tube placement. As previously noted, this bleeding can result in occlusion of the implanted tube after, sometimes quite a while after, the tube has been initially cleared by placement of antibiotic drops. Antibiotics alone or antibiotics in combination with steroids may not alleviate this longer term problem.

### SUMMARY OF THE INVENTION

The invention utilizes topical application of an antibiotic in a therapeutic concentration in combination with a vasoconstrictor in a therapeutic concentration to improve the likelihood that tubes, placed in the eardrum to resolve chronic infection, remain patent and functioning. An ear drop formulation is provided with these two components. The invention addresses simultaneously infections and the tendency of the eardrum to bleed and the resulting clotting to occlude the tube.

### DETAILED DESCRIPTION OF THE PREFERRED EMBODIMENTS

The current invention is an eardrop containing, in addition to an antibiotic to control or eliminate middle ear infection, a vasoconstrictor to stop bleeding of the eardrum at the site of myringotomy incision. In a preferred embodiment the vasoconstrictor is a decongestant. This vasoconstrictor acts directly on the blood vessels in the ear to cause the blood vessels to constrict, thereby reducing the tendency of the tissue near the incision to bleed. The combination of antibiotic and vasoconstrictor has proven superior to drops without a vasoconstrictor in preventing obstruction of the tube.

Clinical experience has shown that when blood is visible at the time of tube placement, application of the combination drop of this invention clearly reduced the likelihood of the tube becoming obstructed with blood. Various embodiments of this combination would include any of various antibiotics used topically to treat the middle or external ear. These embodiments would also include any of various vasoconstrictors. Preferred embodiments include antibiotics with demonstrated lack of toxicity for the inner ear, such as ofloxacin and ciprofloxacin. Gentamycin, tobramycin, and neomycin are effective in treating middle ear infections topically, but there are scattered reports of ototoxicity. Thus, these antibiotics are not used as frequently. Gentamycin and tobramycin come in drop formulation for use as eye drops. Technically use of these drops for the ear is ‘off label use,’ but a clinician may choose to use these antibiotics in the ear topically under certain circumstances. For example, an allergy to the demonstrably non-toxic antibiotics may lead the clinician to use gentamycin or neomycin drops in a particular case after informing patient and/or family of the slight risk of toxicity to the ear.

Some representative combinations included in the most preferred embodiment include
Ciprofloxacin with phenylephrine
Ciprofloxacin with oxymetazoline
Ofloxacin with phenylephrine
Ofloxacin with oxymetazoline.
Neomycin/polymyxin B/hydrocortisone with phenylephrine
Neomycin/polymyxin B/hydrocortisone oxymetazoline
Gentamycin with phenylephrine
Gentamycin with oxymetazoline
Tobromycin with phenylephrine
Tobromycin with oxymetazoline
Corticosporin with phenylephrine
Corticosporin with oxymetazoline

The listed antibiotics are well known in the medical literature as effective in the treatment of middle ear infections. Examples are: Nawasreh OO, Al-Wedyan IA, Prophylactic ciprofloxacin drops after tympanostomy tube insertion. Saudi Med J 2004; 25:38-40 (cipro is effective at preventing post-operative ear drainage after tube insertion); Roland PS, et al., Topical Ciprofloxacin/Dexamethasone is superior to Ciprofloxacin alone in pediatric patients with acute otitis media and otorrhea through tympanostomy tubes. Laryngoscope; 113: 2116-2122. (Dexamethosone is a steroid that is sometimes used to reduce inflammation.) Results from these antibiotics are, in the inventor's experience, improved when a tube permits the drops to access the middle ear.

Gentamycin and neomycin can be used for topical application in some circumstances, as mentioned above. For example, Morpeth JF, et al., A comparison of cortisportin and ciprofloxacin otic drops as prophylaxis against post-tympanostomy otorrhea. Int J Ped Otorhinolaryngol 2001;61:99-104, supports use of the identified antibiotics but suggests avoiding ototoxic antibiotics. Further the American Academy of Otolaryngology recommends using the non-ototoxic antibiotics for this purpose unless drug sensitivities force consideration of the ototoxic species. The appropriate therapeutic range for gentamycin range is 0.05 to 0.5% and for tobramycin the range is also 0.05 to 0.5%. For neomycin the appropriate therapeutic range is 1 to 5 mg/ml.

In the preferred embodiments, concentration of stock ofloxacin is 0.3%. Clinical experience supports a therapeutic concentration range of 0.03% to 3% and preferably 0.1 to 3%. Ciprodex, a combination of ciprofloxacin and dexamethasone, has a stock concentration of 3 mg/ml and clinical experience supports a therapeutic range of of 0.3 mg/ml to 10 mg/ml for this combination. Ciprofloxacin has a therapeutic range also of 0.03% to 3% and preferably 0.1 to 3%. For phenylephrine, the range is about 0.1% to 1.0% and preferably 0.125% to 0.75%. For oxymetazoline the concentration range preferred is 0.01% to 0.5%.

In vivo efficacy of this invention has been preliminarily established clinically. Eleven patients were treated with a combination of ofloxacin and oxymetazoline at the time of tube placement. The invention was applied specifically in cases where blood was seen at the time of tube placement, a situation in which the chance of tube occlusion is very high. None of these patients showed post-operative occlusion of myringotomy tubes. In one representative example, a three year-old was undergoing tube placement for recurrent infections. Seconds after making the tiny incision in the eardrum (myringotomy), a drop of blood formed on the surface of the eardrum. While small in amount, this blood would have easily caused obstruction of the tube (the tube having a typical lumen diameter of one millimeter). Ofloxacin drops were combined with oxymetazoline in a 1:1 ratio (final concentrations of 0.15% and 0.025% respectively) and applied to the ear topically. At follow-up one week later, the tube was patent and there was no visible blood in the canal.

Safety of vasoconstrictors for use in the ear has been demonstrated in animal studies. The inventor conducted animal studies involving application of alpha agonists, including phenylphrine (and other similar agents) directly in the middle ear space of guinea pigs. In these studies, phenylephrine at a concentration of 0.05 to 1.0% was applied directly to the round window membrane of the animal. During these studies, compound action potential (CAP)(a measure of inner ear hearing function) was not found to be adversely affected in even a single case after trial on more than 50 animals. The monitoring of CAP was not specifically reported in published reports, but rather was monitored on a routine basis as a part of the experimental setup. Further, none of the patients treated with the oxymetazoline/ofloxacin combination had any adverse effect on hearing. See also Isaacson G et al., Oxymetazoline solutions inhibit middle ear pathogens and are not ototoxic. Ann Otol rhinol Laryngol 2005;1 14:645-51 (oxymetazoline is non-toxic to the ear in guinea pigs).

I claim:

1. A pharmaceutical composition for treating or preventing occlusion of myringotomy tubes and middle ear infections comprising:
a) an antibiotic in a therapeutically effective concentration; and
b) a vasoconstrictor in a therapeutically effective concentration.

2. The composition of claim 1 in which the antibiotic is selected from the group consisting of ciprofloxacin, ofloxacin, polymyxin, B/hydrocortisone.

3. The composition of claim 2 in which the antibiotic is selected from combinations of the members of the group.

4. The composition of claim 1 in which the antibiotic is selected from the group consisting of gentamycin, neomycin, tobromycin.

5. The composition of claim 1 in which the vasoconstrictor is a decongestant.

6. The composition of claim 5 in which the decongestant is selected from the group consisting of oxymetazoline and phenylephrine.

7. The composition of claim 2 in which the therapeutic range of the selected antibiotic is 0.03% to 3% for ciprofloxacin and ofloxacin.

8. The composition of claim 4 in which the therapeutic range of the selected antibiotic is 0.05% to 0.5% for gentamycin and tobramycin.

9. The composition of claim 6 in which the therapeutic range of the selected decongestant is 0.1% to 1.0% for phenylephrine or 0.01% to 0.5% for oxymetazoline.

10. A method of preventing occlusion of myringotomy tubes comprising the steps of
a) compounding a mixture of therapeutically effective concentrations of an antibiotic and a vasoconstrictor;
b) applying the mixture in drops to the ear canal, the region of the tube, and through the tube.

11. The method of claim 10 in which the antibiotic is selected from the group consisting of ciprofloxacin, ofloxacin, polymyxin, B/hydrocortisone, gentamycin, tobramycin, and neomycin.

12. The method of claim 10 in which the vasoconstrictor is a decongestant.

13. The method of claim 10 in which the decongestant is selected from the group consisting of oxymetazoline and phenylephrine.
